(19) United States
(12) Patent Application Publication
(10) Pub. No.: US 2008/0097549 A1
(43) Pub. Date: Apr. 24, 2008

(54) Electrode Assembly and Method of Using Same

(76) Inventors: Michael E. Colbaugh, Trafford, PA (US); William Clegg, Gibsonia, PA (US); Matthew Sass, Pittsburgh, PA (US)

Correspondence Address: MICHAEL W. HAAS;RESPIRONICS, INC., 1010 MURRY RIDGE LANE, MURRYSVILLE, PA, 15668, US

(21) Appl. No.: 11/846,895

(22) Filed: Aug. 29, 2007

Related U.S. Application Data
(60) Provisional application No. 60/841,802, filed on Sep. 1, 2006.

(51) Int. Cl. A61N 1/00

(52) U.S. Cl. 607/55; 607/2

(57) ABSTRACT

An electrode assembly for delivering electrical stimulation to a vestibular system of a user or monitoring a physiological parameter of the user. The apparatus includes a body member comprising an electrode support portion and a curved portion extending from the electrode support portion, and an electrode coupled to the electrode support portion. A method of using such an apparatus is also disclosed. The present invention also provides an electrode assembly that includes a body member, an electrode coupled to the body member, a hydrogel element disposed on at least a portion of the electrode. In one embodiment, the hydrogel element includes a hydrogel material and a insulating material disposed on or in the hydrogel material such that the insulating layer disperses energy transmitted by the electrode through the hydrogel material.

### CROSS-REFERENCE TO RELATED APPLICATIONS

This application claims priority under 35 U.S.C. § 119(e) from provisional U.S. patent application No. 60/841,802, filed Sep. 1, 2006, the contents of which are incorporated herein by reference.

### BACKGROUND OF THE INVENTION

1. Field of the Invention

The present invention relates to an electrode assembly for stimulating the user, such as user's vestibular system and/or monitoring the user, and, in particular, to an electrode assembly having a body configured to fit around the external ear and an electrode coupled to the body member and disposed so as to deliver energy to the user, such as the vestibular system and/or monitor the user, and to a method of using such an apparatus.

2. Description of the Related Art

The vestibular system is responsible for the detection of the position and motion of the head in space. The semicircular canals, which are located in the inner ear, are the sensory organs of the vestibular system, and collect head position and motion information and transmit it to the central nervous system via the eighth cranial nerve. Disorders of the vestibular system may result in physiological disorders such as dizziness, vertigo, and nausea, with symptoms ranging in severity from mild to completely debilitating.

Stimulation of either the semicircular canals, the utricle, saccule, or other otolith organs, as well as stimulation of the nerve fibers leading from these organs or the eighth cranial nerve, or combinations thereof, result in a sensation of movement in normal subjects. Moderate stimulation of the vestibular system may cause perceptions of mild movement that are not unpleasant, but can have beneficial properties, such as promoting a sleep state in a patient.

Many techniques for stimulating the vestibular system exist. These methods include calorimetric, chemical, and electrical approaches. Calorimetric and chemical stimulation typically take the form of direct application of a warm solution or a chemical compound, either directly or indirectly, to the eighth cranial nerve. Electrical stimulation of the vestibular system typically includes the placement of an electrode on the surface of the skin, e.g., over the mastoid bone behind the ear, or the piercing of the tympanic membrane with an electrode for direct stimulation of the semicircular canals. It is also known to stimulate the vestibular system by invasive electrodes implanted within the inner ear.

Conventional surface electrodes that are used to stimulate the vestibular system are not optimized for patient comfort. They typically consist of a patch-type electrode that is applied on the surface of the patient, typically near the mastoid. This type of electrode includes an adhesive layer that is strong enough to secure to the electrode to the surface of the user. Naturally, there are undesirable consequences of using such an adhesive. For example, removing the electrode requires detaching the adhesive from the surface of the user, which can be a painful process, especially when the adhesive is secured to hair.

In addition, it is difficult for a layperson to apply a surface electrode to the precise location where stimulation is to be provided. Properly position in the patch-type electrode is even more difficult when the user is attempting to apply the electrode to himself or herself, due to the fact that the side of the head is not easily reached by the user and not easily visualized. If the surface electrode is not properly positioned on the desired location of the user, the stimulation energy provided by the surface electrode may cause unwanted consequences, such as the non-specific activation of facial muscles, involuntary twitches, auditory miss-perceptions, or modulation of the nerves in the carotid/sinus region of the neck.

It is also known to stimulate the vestibular system using an electrode disposed in the ear canal. However, users may not prefer having an object in their ear during the stimulation therapy, which in some situations is administer while the user is falling asleep and/or is sleeping. Indeed, it is believed that about 15-30% of the population dislikes having anything in their ear.

Thus, a long standing need exists within the medical field for a system that allows for the specific and non-invasive activation of the vestibular system that is comfortable enough for a patient to wear for an extended period time, such as during sleep, and during periods of quiet resting. The system should be easy for the user himself/herself to position the stimulation electrode on the head in the proper position and without assistance. In addition, such a system should allow for the delivery of a wide variety of stimulation frequencies and waveforms to the vestibular system of the inner ear.

The system should also be comfortable for the patient even when the system is disposed between the patient and an underlying support, for example when the patient's head is lying on a pillow with the stimulation system situated between the head and pillow. In addition, the system should remain firmly in place, so that the stimulating energy is delivered to the desired anatomical location despite normal movement and contact forces, such as tossing and turning during sleep and when attempting to fall asleep.

### SUMMARY OF THE INVENTION

Accordingly, it is an object of the present invention to provide a vestibular stimulation system that overcomes the shortcomings of conventional vestibular stimulation systems. This object is achieved according to one embodiment of the present invention by providing a vestibular stimulation system that includes an electrode assembly for delivering electrical stimulation to a vestibular system of the user. The electrode assembly includes a body member having an electrode support portion and a curved portion extending from the electrode support portion. The curved portion allows the electrode assembly to be positioned around the back of the external ear so that the electrode support portion is disposed at the proper stimulation application location on the user when the electrode assembly is coupled to the user. An electrode coupled to the electrode support portion delivers the stimulation energy.

It is yet another object of the present invention to provide a method of providing stimulation energy to the vestibular system that does not suffer from the disadvantages associated with conventional vestibular stimulation techniques. This object is achieved by providing a method that includes securing an electrode assembly to a surface of a user by wrapping at least a portion of the electrode assembly around at least a portion of a perimeter at a back of the human ear and providing an energy delivery portion proximate to a mastoid. Stimulation energy is provided to the electrode assembly.

In a further embodiment, this object is achieved by providing a method of delivering electrical stimulation to a user that includes providing an electrode assembly, applying a hydrogel element to a portion of the electrode assembly, affixing the electrode assembly to a user, using the electrode assembly to deliver energy to such a user, removing the hydrogel element from the electrode assembly, applying a subsequent hydrogel element to the portion of the electrode assembly, and reusing the electrode assembly to deliver energy to such a user.

In another embodiment the present invention contemplates providing an electrode assembly that includes a body member, an electrode coupled to the body member, a hydrogel element disposed on at least a portion of the electrode. The hydrogel element includes a hydrogel material, and a insulating material disposed on or in the hydrogel material such that the insulating layer disperses energy transmitted by the electrode.

In a still further embodiment, the present invention contemplates using the above-described systems, methods, and apparatus to monitor a physiological parameter of the user in addition to or instead of delivering energy to the user.

While the electrode assembly has been described above as being used to simulate the vestibular system, it is a further object of the present invention to provide an electrode assembly and method of using same, as described above, to stimulate other portions of the user, in addition to or instead of the vestibular system.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, an and “the” include plural referents unless the context clearly dictates otherwise.

### BRIEF DESCRIPTION OF THE DRAWINGS

FIG. 1 is a rear perspective view of a first embodiment of an electrode assembly according to the principles of the present invention;

FIG. 2 is an exploded view of the spiral electrode of FIG. 1;

FIG. 3 is a side view of a human head showing the location of the electrode assembly of FIG. 1 on the user;

FIG. 4 is a front perspective of view of a second embodiment of n electrode assembly according to the principles of the present invention;

FIG. 5 an exploded view of the spiral electrode of FIG. 4;

FIG. 6 is a detailed front perspective view of a portion of the electrode assembly of FIG. 4;

FIGS. 7A and 7B are front perspective views illustrating the technique for attaching an adhesive assembly to a body member;

FIG. 8 is a rear perspective view of a third embodiment of an electrode assembly according to the principles of the present invention;

FIG. 9 is a front perspective view of the body member in the electrode assembly of FIG. 8;

FIG. 10 is a front perspective view of the electrical lead in the electrode assembly of FIG. 8;

FIG. 11 is a front perspective view showing the attachment of the electrical lead of FIG. 10 to the body member of FIG. 9 in the electrode assembly of FIG. 8;

FIG. 12 is a rear perspective view showing the attachment of an adhesive member to the body member as a step in the manufacturing process for the electrode assembly of FIG. 8;

FIG. 13 is a rear perspective view showing the application of a conductive member to the body member and the adhesive member as step in the manufacturing process for the electrode assembly of FIG. 8;

FIG. 14 is a rear perspective view showing the application of a patient contacting pad to the remaining portions of the electrode assembly during the manufacturing process for the electrode assembly of FIG. 8;

FIG. 15 is an exploded view if a fourth embodiment of an electrode assembly according to the principles of the present invention;

FIG. 16 is an exploded view if a fifth embodiment of a electrode assembly according to the principles of the present invention;

FIG. 17 is a front perspective of illustrating a pair of electrode assemblies connected to one another via a strap;

FIG. 18 is a rear perspective view of a sixth embodiment of an electrode assembly according to the principles of the present invention;

FIG. 19 is a rear perspective view illustrating the assembly of the body member in the electrode assembly of FIG. 18 and a detailed view of a portion of the body member;

FIG. 20 is a front perspective view illustrating the body member in the electrode assembly of FIG. 18;

FIG. 21 a rear perspective view illustrating the assembly of an electrode with the body member in the assembly of the electrode assembly of FIG. 18;

FIG. 22 is a front perspective view of the electrode illustrated in FIG. 21;

FIG. 23 is a front perspective view illustrating the attachment of an electrical lead to the body member in the assembly of the electrode assembly of FIG. 18;

FIG. 24 is a front perspective view illustrating the assembly of a protective cover to the body member in the assembly of the electrode assembly of FIG. 18;

FIG. 25 is a front perspective view illustrating a portion of the assembled electrode assembly of FIG. 18;

FIG. 26 is a cross-sectional front perspective view illustrating the connection of the electrical lead to the electrode of FIG. 22 and the cover shown in FIG. 24;

FIG. 27A is an exploded view showing the attachment of an adhesive assembly the electrode assembly of FIG. 18;

FIG. 27B is an exploded view showing an alternative configuration for the adhesive assembly suitable for use in the electrode assembly of FIG. 18;

FIG. 28 is a perspective of a plurality of adhesive assemblies suitable for use with the electrode assembly of FIG. 18 disposed on a common backing sheet;

FIG. 29 is a rear exploded view illustrating the assembly of a seventh embodiment of an electrode assembly according to the principles of the present invention;

FIGS. 30A-30E are rear views illustrating various embodiments for the insulating layer disposed over the electrode/conductive layer suitable for use in the electrode assembly of FIG. 28; and

FIG. 31 is a rear exploded view showing the attachment of an adhesive assembly to the electrode assembly of FIG. 29.

### DETAILED DESCRIPTION OF THE EXEMPLARY EMBODIMENTS

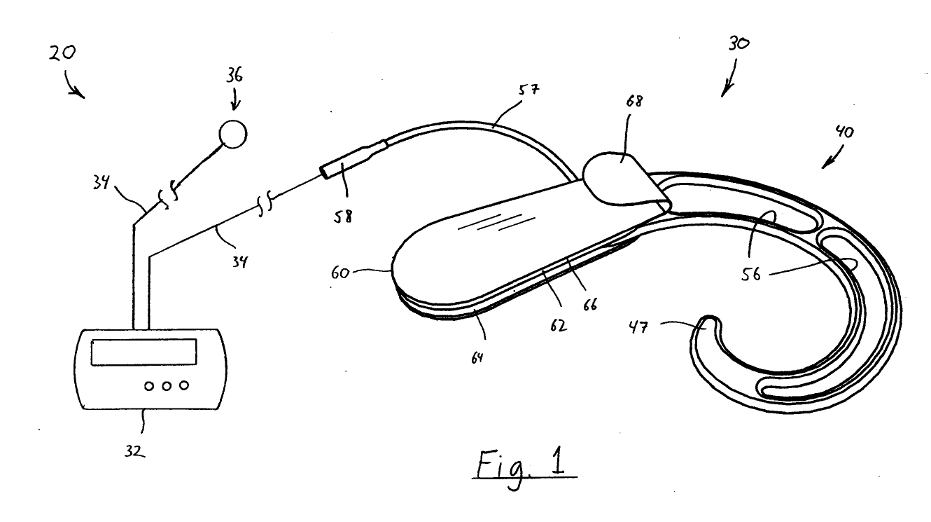

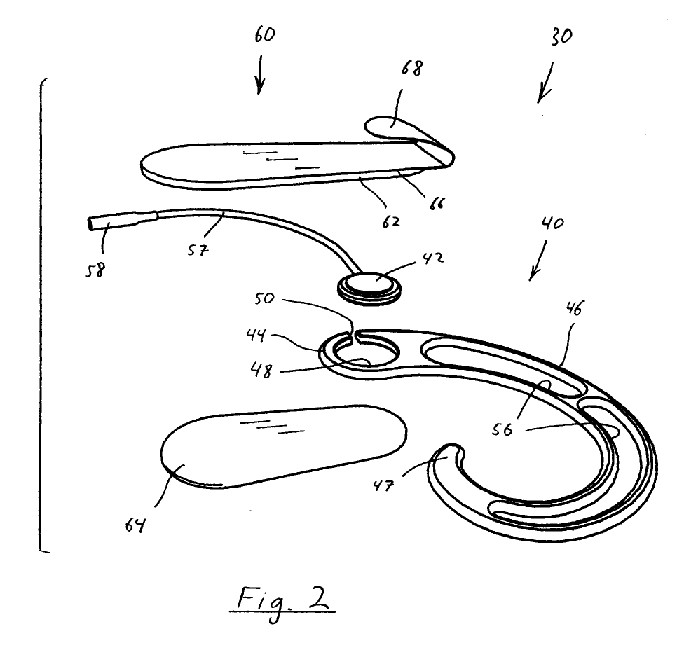

FIGS. 1-3 illustrate a vestibular stimulation system 20 for delivering electrical stimulation to a vestibular system of a user. In particular, these figures illustrate a first embodiment of an electrode assembly 30 according to the principles of the present invention for use in the vestibular stimulation system. Details of electrode assembly 30 and alternative embodiments thereof, are provided below. In addition to electrode assembly 30, vestibular stimulation system 20 includes a stimulating energy source/controller 32 to which the electrode assembly is coupled.

Source/controller 32 provides energy, typically in the form or an electrical current to electrode assembly 30. Source/controller 32 includes a controller and an energy source, with the controller controlling the delivery of energy from the energy source to the electrode assembly, thereby controls the stimulation energy or therapy being delivered to the user via the electrode assembly. In its most simple form, the controller can be a manually actuated device, for example and on/off switch. In more sophisticated embodiments, the controller regulates the amount of energy and/or the pattern of energy delivered by source 32 to electrode assembly 30. The controller can include a processor, circuit, or other electrical/electro-mechanical device that controls the energy provided to the electrode assembly. A wire or electrical lead 34 couples the energy source to the electrode assembly. The energy source can be powered by AC power supply or a DC power supply, such as one or more batteries. Of course, controller/source 32 includes the necessary circuits, input/output devices, programming, and other features to perform the function of providing electrical energy to the electrode assembly.

It can be appreciated that in order to provide an electrical stimulation to the user, at least two electrodes must be located on the user. In the embodiment shown in FIG. 1, a second electrode assembly 36 is schematically illustrated that is also coupled to controller/source 32. Of course, more than two electrode assemblies can be attached to the energy controller/source. Second electrode assembly 36 can have the same configuration as electrode assembly 30 or can have a different configuration.

Vestibular stimulation system 20 can be used to provide stimulation to the vestibular system to achieve any result known to those skilled in the art. For example, U.S. Pat. No. 6,748,275 teaches providing energy to the vestibular system to induce a rocking sensation thereby promoting the onset of sleep and to contract the upper respiratory muscles.

Electrode assembly 30 includes a body member, generally indicated at 40, and an electrode 42 coupled to the body member. Body member 36 includes an electrode support portion 44 and a curved portion 46 extending from the electrode support portion. Electrode 38 is coupled to electrode support portion 40 of the body member. More specifically, in this embodiment, electrode 42 is a spot electrode that is substantially flat has a generally circular or elliptical shape. Electrode 42 is formed from any suitable conductor or combination of materials. Body member 40 can be formed from a variety of materials or combination of materials. In the illustrated exemplary embodiment, the body member is formed from a semi-rigid or a flexible material so that the body member, and, in particular, curved portion 46 can flex to fit the user, as discussed in greater detail below.

Electrode 42 and electrode support portion 44 are configured such that the electrodes mounts onto the body member. To this end, electrode support portion 44 includes an opening 48 sized and configured to receive the electrode. A notch 50 is provided so that the electrode support portion can flex to receive the electrode and return to it original shape once the electrode is disposed in opening 48, thereby snuggly affixing the electrode to the body member. The electrode can be detached from the body member simply by snapping it out the opening.

Curved portion 46 of body member 40 is shaped in the form of a semi-circle, spiral, ovoid, or ellipse because these shapes generally correspond to the shape of the back of the human ear. As shown in FIG. 3, the external portion of the human ear includes an auricula or pinna 52, which is the cartilage that protrudes from the surface of the head. The crease at the back of the ear where the pinna extends from the head or skull, and which is generally indicated at 54, is irregularly concave. Curved portion 46 of body member 40 is shaped to generally match this portion of the human anatomy so that the curved portion rests at or near this crease. That is, the shape of the curbed portion mimics the ear's shape to provide a natural curve fit of the electrode assembly to the back of the ear.

The curved portion is located at the crease at the back of the ear so that the electrode portion of the electrode assembly can be properly located on the user, namely at, on, or near the mastoid process, by someone with little or no knowledge of human anatomy. The mastoid bone immediately behind the ear, provides a relatively large target area for providing vestibular stimulation. If a patch-type of electrode assembly is used, a user who knows little of the human anatomy, will likely have difficulty properly position the electrode on the mastoid. This is exacerbated by the face that it is difficult to see the side of the head as you attempt to apply an electrode to the side of the head. The present invention solves this problem by using the back of the ear as an anchor location or point of reference so that the user can place the electrode at the desired location merely by arranging the curved portion at the back of the ear.

Curved portion also helps secure the electrode assembly to the user by wrapping, as least somewhat, around a portion of the external ear. The top of the spiral forms a comfortable anchor point for the ear contact structure, and somewhat automatically finds the proper place to “hook” the ear at the top of the ear. That is, in an exemplary embodiment, a distal end 47 of curved portion 46 of body member 40 hooks over the tip or top of the ear. By doing so, the electrode assembly is easily located in the user and held in place on the ear. The electrode assembly has a generally flat configuration so that the user can lie on it during use, for example as the electrode assembly delivers stimulation to the vestibular system to induce or promote sleep.

In the illustrated embodiment, curved portion 46 includes cutouts 56. These are provided to reduce the amount of material that must be attached to the user, allow the surface of the user under the electrode assembly to breathe, and provide some flexibility for the curved portion. It is to be understood that the shape, size, number, and configuration for cutouts 56 can be varied as desired.

Electrical lead 34 can have any desired length and can coupled to electrode 42 in any conventional manner. In the illustrated embodiment, it is relatively short, e.g., 0.5-4.0 inches and includes a connection lead 57 and a connection terminal 58 at the end of the connection lead. This configuration is advantageous in that it allows electrode assembly to be provided as a relatively small unit that can be disposable, while the rest of the electrical lead can be reused with another electrode assembly. Connection terminal 58 can have any suitable configuration.

An adhesive assembly 60 is provided to attach the electrode assembly to the user. In this embodiment, adhesive assembly 60 includes an adhesive member 62, an adhesive support 64 disposed on one side of the adhesive member, and a removable backing 66 disposed on another side of the adhesive member. A further layer of adhesive (not shown) may be provided on adhesive support 64 so that any portion of the adhesive support not contacting adhesive member 62 will adhere to the electrode assembly. Removable backing 66 includes a tab 68 to allow the user to grip the backing and peel it off of member layer 62. The size, shape, and configuration for the adhesive assembly, as well as the individual components thereof, can be varied. In addition, multiple adhesive assemblies can be provided in one electrode assembly.

The present invention contemplates that adhesive member 62 is any suitable adhesive, an example a suitable adhesive is a hydrogel. In this illustrated embodiment, the adhesive member overlies at least a portion of electrode 42. In which case, the portion of the adhesive member should be electrically conductive so that energy can be transmitted to the patient from the electrode through the adhesive member. An electrically conductive adhesive is further advantageous in that it ensures a good electrical contact with the surface of the user.

It is to be further understood that the adhesive member need not be provided over the electrode, leaving the electrode (or portions thereof) in direct contact with the surface of the user. In which case, the adhesive need not be electrically conductive. Adhesive support 64 can also any material or combination of material that supports for the adhesive member. An example of a suitable material is a strip of cloth or foam.

This embodiment of electrode assembly 30 provides two alternative ways of using, and reusing, portions of the electrode assembly. In one version, as noted above, the entire electrode assembly is disposable after one or more uses. The user need only detached the electrode assembly from electrical lead 34 by decoupling it via connection terminal 58. The electrode assembly is disposed of and a new one is attached to the electrical lead, allowing most of the electrical lead to be reused. This also allows electrode assemblies of different configurations to be used with the same electrical lead 36 and source/controller 32.

In another version, only the portion of the electrode assembly that need to be replaced after one or more uses, namely adhesive assembly 60, is disposable. After the adhesive is no longer capable of sufficiently bonding to the user, the adhesive assembly can be removed from body member 40 and electrode 42 simply by removing adhesive member 62 and backing 66. Adhesive support 64 can also be removed, but depending on the type of material used for the adhesive support, it may be possible to reuse this as well. A new adhesive member 62 (and adhesive support if necessary) is applied to the electrode body and the entire electrode assembly is now ready for use, without having to have disposed of the electrode or body member.

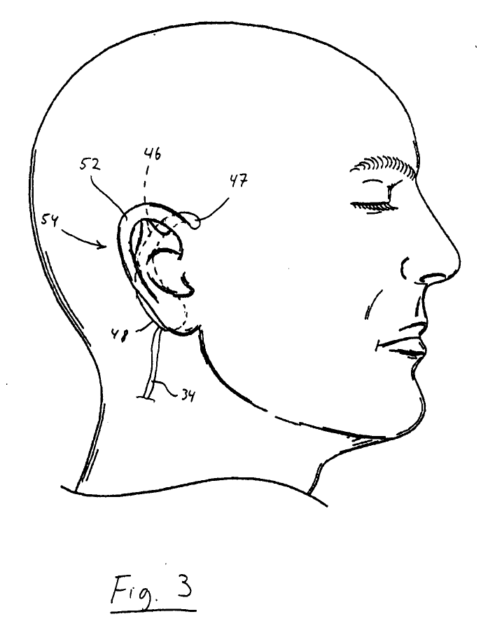

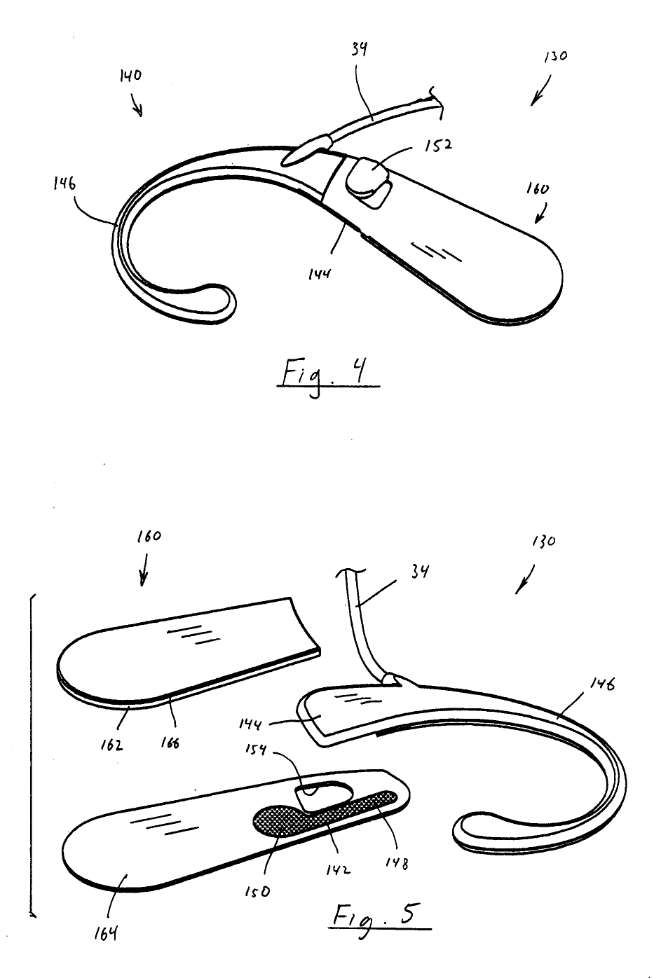

FIGS. 4-7B illustrate a second embodiment for electrode assembly 130 according to the principles of the present invention. Electrode assembly 130 includes a body member 140 and an adhesive assembly 160. Body member 140 includes an electrode support portion 144 and a curved portion 146. In an exemplary embodiment, curved portion 146 is formed from a hard, molded plastic, such as the Polycarbonate GE Lexan HP1 having a thickness of approximately 0.040 inch. For comfort, the curved portion can include a soft material, such as a soft elastomer, overmolded to the hard plastic portion.

The electrode support portion is the portion of the body member to which electrode assembly 130 attaches. The electrode support portion includes an attachment assembly for selectively coupling the electrode assembly to the body member and for providing an electrical connection between electrical lead 34 attached to the body member and electrode 142 provided in the adhesive assembly.

Adhesive assembly 160 includes an adhesive member 162 and an adhesive support 164. In an exemplary embodiment, adhesive support is a stamped from a thin, flexible material. As in the previous embodiment, the adhesive member and an adhesive support can have a variety of sizes and shapes and can be made from one or more of a variety of materials. A removable backing 166 can also be provided over the adhesive member.

In this embodiment, an electrode 142 is associated with the adhesive assembly, rather than being attached directly to the body member as in the previous embodiment. More specifically, electrode 142 is disposed on adhesive support 164, for example by being printed or coated on the material forming adhesive support 164. Electrode 142 includes a connection portion 148 and a main portion 150. The main portion is covered by adhesive member 162 leaving connection portion 148 exposed.

In the illustrated exemplary embodiment, the attachment assembly, which couples adhesive assembly 160 to body member 140, includes a protrusion 152 provided at electrode support portion 144 and an corresponding opening 154 provided in adhesive assembly 160. Of course, the present invention also contemplates reversing the orientations of these components so that the protrusion is provided on the adhesive assembly and the opening is provided in the body member.

To provide an electrical connection between the electrical lead 34 and electrode 142, an contact terminal 156 is provided at the end of the electrical lead. As best shown in FIG. 6, the contact terminal is coupled to the body member so as to extend slightly therefrom. When adhesive assembly 160 is coupled to body member 140, contact terminal 156 makes electrical contact with connection portion 148 of electrode 42. The overlying portions of the body member, and the electrode support portion, in particular, are shown in dashed lines in FIG. 6.

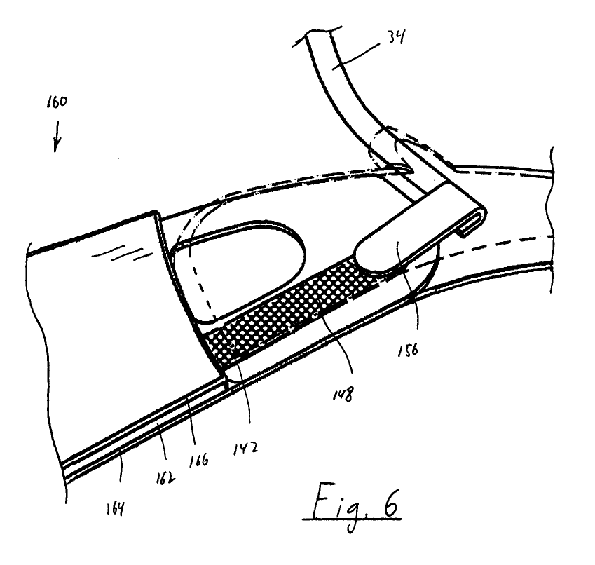

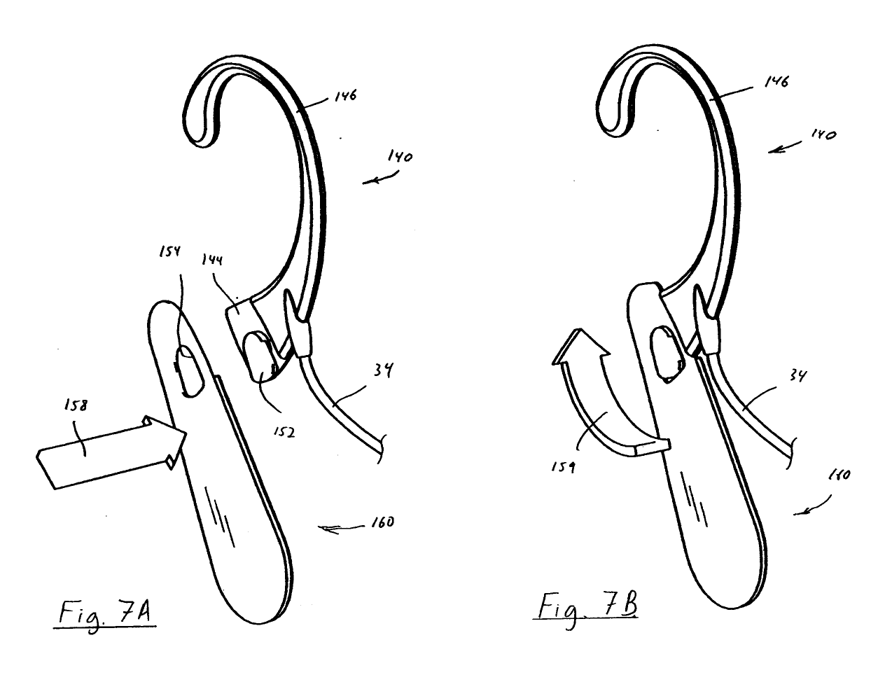

This embodiment allows adhesive assembly 160 and body member 140 to be manufactured and sold as separate elements. Thus, a user need only have one body member, and a supply of adhesive assemblies in order to use the stimulation system many times, reusing the body member and replacing the adhesive assembly when appropriate. Assembling the adhesive assembly with the body member is shown in FIGS. 7A and 7B. This is a simple, two-step process. First, the adhesive assembly is oriented at an angle with respect to the body member and is moved into contact with the body member, as indicated by arrow 158 so that protrusion 152 enters opening 154.

Protrusion 154 is configured with an undercut at its base such that rotating the adhesive assembly relative to the body member, as indicated by arrow 159, causes an edge of opening 154 to be trapped under the undercut, thereby securing the adhesive assembly to the body member. The assembled electrode assembly is shown in FIG. 4.

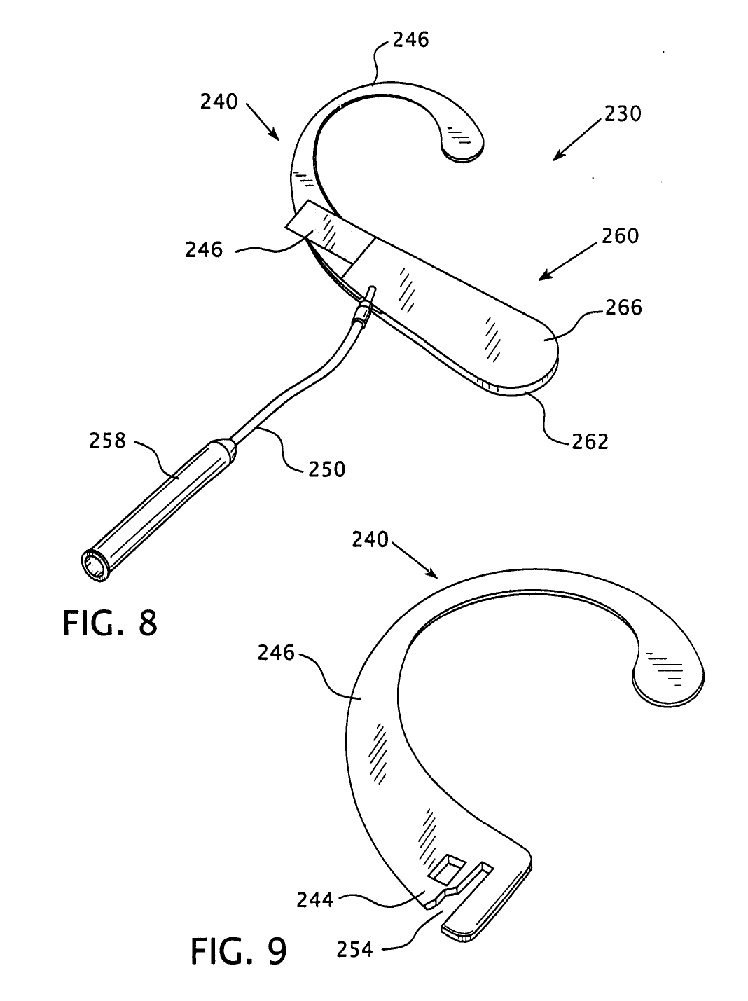

FIGS. 8-14 illustrate a third embodiment for electrode assembly 230 according to the principles of the present invention. Electrode assembly 230 includes a body member 240 and an adhesive assembly 260. Body member 240 includes an electrode support portion 244 and a curved portion 246. In this embodiment, the body member is defined from a unitary piece of material, such as polycarbonate. A connection lead 250 attaches to the electrode support portion.

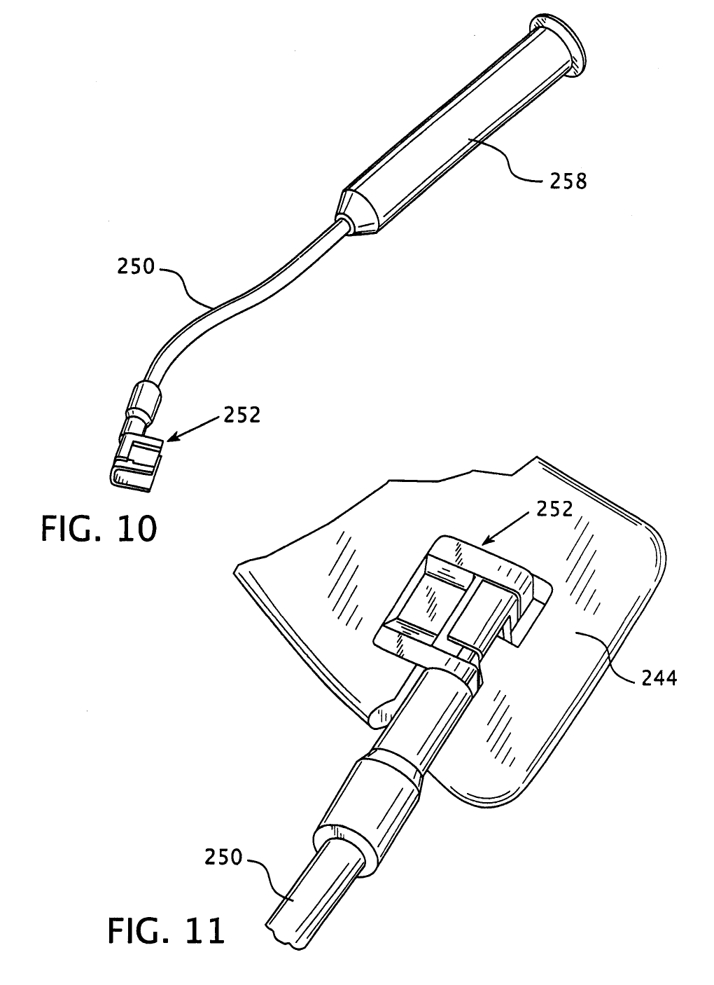

As perhaps best shown in FIGS. 10 and 11, connection lead 250 includes a first connection terminal 252 that inserts into to a slot 254 provided in the electrode support portion of the body member. In an exemplary embodiment, one or more locking pins are provided in the slot, the connection terminal, or both to prevent the connection terminal from being removed from the slot. First connection terminal 252 also functions as a point of contact with an electrode 242 as discussed in detail below. Connection lead 250 is relatively short and includes a second connection terminal 258 that selectively connects to an electrical lead using any suitable connection configuration.

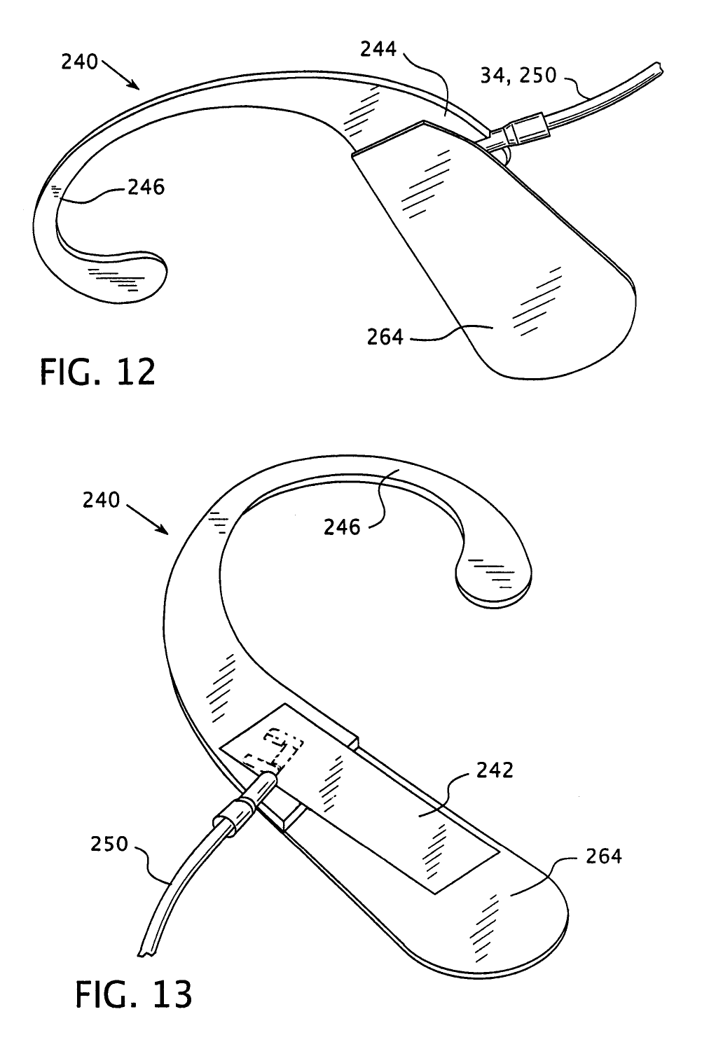

After first connection terminal 252 is provided in slot 254, adhesive assembly 260 is coupled to body member 240. See FIG. 12. This as accomplished by first applying an adhesive support 364 on one side of the body member such that the adhesive support is attached to electrode support portion 244 of the body member. The present invention contemplates that any suitable connection technique can be used to couple adhesive support 264 to electrode support portion 244. In an exemplary embodiment, adhesive support 264 is a medical tape, such as the MED 5322P medical tape sold by Avery Dennison.

An electrode 242 is then applied over electrode support portion 244, over at least a portion of adhesive support 264, and in electrical contact with first connection terminal 252. In the illustrated embodiment, electrode 242 is a conductive strip having a generally rectangular configuration. An example of a suitable material for the electrode is the 9713 X-Y-Z tape manufactured by 3M. It is to be understood that the present invention contemplates that electrode 242 can be any suitable size or shape, can be located at other locations, and can include multiple electrodes electrically connected to one another, for example.

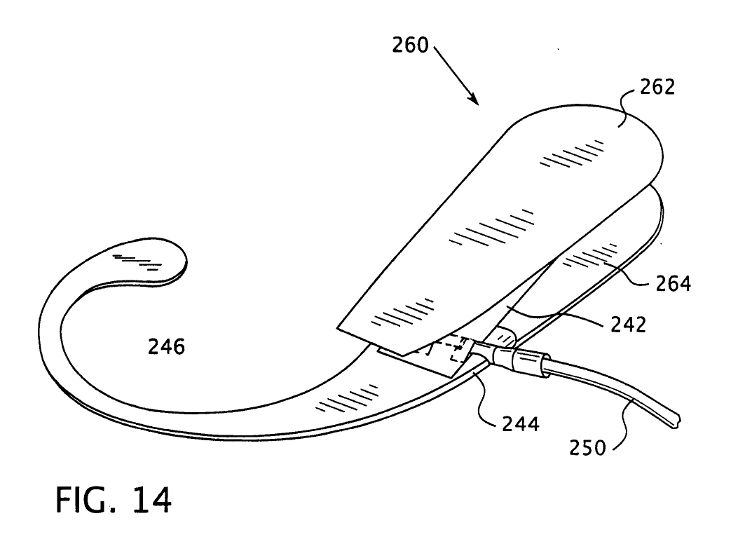

Next, an adhesive member 262 is disposed over electrode 242. See FIG. 14. In the illustrated exemplary embodiment, adhesive member 262 has a shape that generally corresponds to that of adhesive support 264 so that the electrode 242 is completely sandwiched between these two components. It should be understood, however, that the present invention contemplates other configurations for the adhesive member. An example of a material suitable for use as adhesive member 262 is the AG603 hydrogel sold by Amgel® Technologies.

In an exemplary embodiment, a backing 266 is pre-attached to adhesive member 262 so that both the backing and the adhesive member are attached to the electrode assembly as one. Of course, a backing can be applied over all, or a portion of, adhesive member 262 after the adhesive member is affixed to the electrode assembly. Alternatively, the backing can be omitted entirely. Backing 266 includes an optional tab 268 to make it easy for the user to removing the backing and expose the adhesive member for applying the electrode assembly on his or her head.

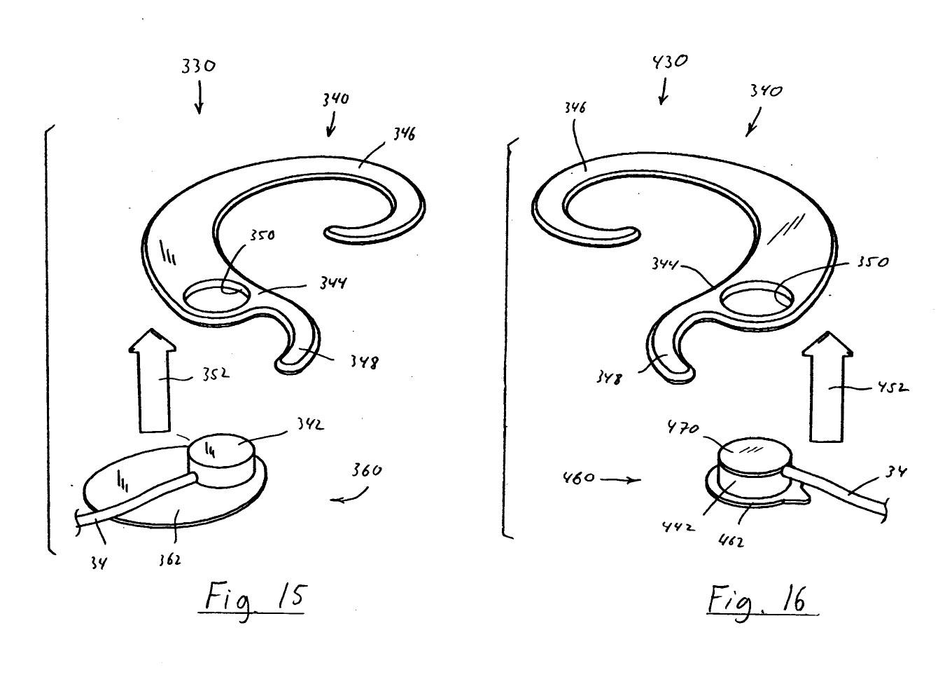

FIG. 15 illustrates a fourth embodiment for an electrode assembly 330 according to the principles of the present invention. Electrode assembly 330 includes a body member 340 and an adhesive assembly 360. Body member 340 includes an electrode support portion 344 and a curved portion 346. An extension 348 is provided from an end of the body member to make it easy to grip and to further extend the body member around the back of the human ear. Electrode support portion 344 includes an opening 350 to receive a portion of adhesive assembly 360.

Adhesive assembly 360 of this embodiment includes an electrode 342 and an adhesive member 362 attached to the electrode. Electrode 342 is sized and configured to be received in opening 350 as indicated by arrow 352 such that adhesive member 362 is disposed between the surface of the user and electrode 342. In the illustrated embodiment, there is some degree of flexibility in how the adhesive assembly is oriented relative to body member 340 so that the relative position of adhesive member 362 can be controlled. However, the position of electrode 342 relative to the body member does not change because of it being fixed relative to the body member, i.e., at opening 350.

While electrode 342 is shown as being generally cylindrical and opening 350 is shown as being generally circular, it is to be understood that other configurations are contemplated for these components of the electrode assembly. Adhesive member 350 is a hydrogel element that is sufficiently self-supporting so as not to need an adhesive support. Of course, the present invention contemplate including an adhesive support so long as it does not interfere with the electrical connection between the electrode and the surface of the user. Also, a backing (not shown) can be provided over one or both surfaces of adhesive member 362.

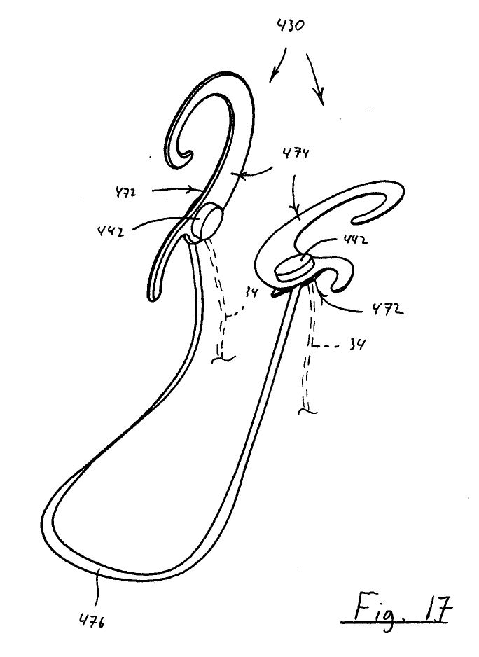

FIGS. 16 and 17 illustrate a fifth embodiment for an electrode assembly 430 according to the principles of the present invention. Electrode assembly 430 includes a body member 340, which is identical to that of the previous embodiment, and an adhesive assembly 460. Adhesive assembly 460 includes a electrode 442, an adhesive member 462, and a cover member 470. Adhesive assembly 460 inserts into opening 350 in body member 340 as indicated by arrow 452. Adhesive assembly 460 is configured such that adhesive member 462 is disposed between the surface of the user and electrode 442. That is, a first side 472 of electrode assembly is adjacent the skin of the user, and a second side 474 is spaced apart from the surface of the user on which the electrode assembly is mounted. As shown in FIG. 17, a connection strap 476 is provided to couple the two electrode assembly together so that they can be easily managed.

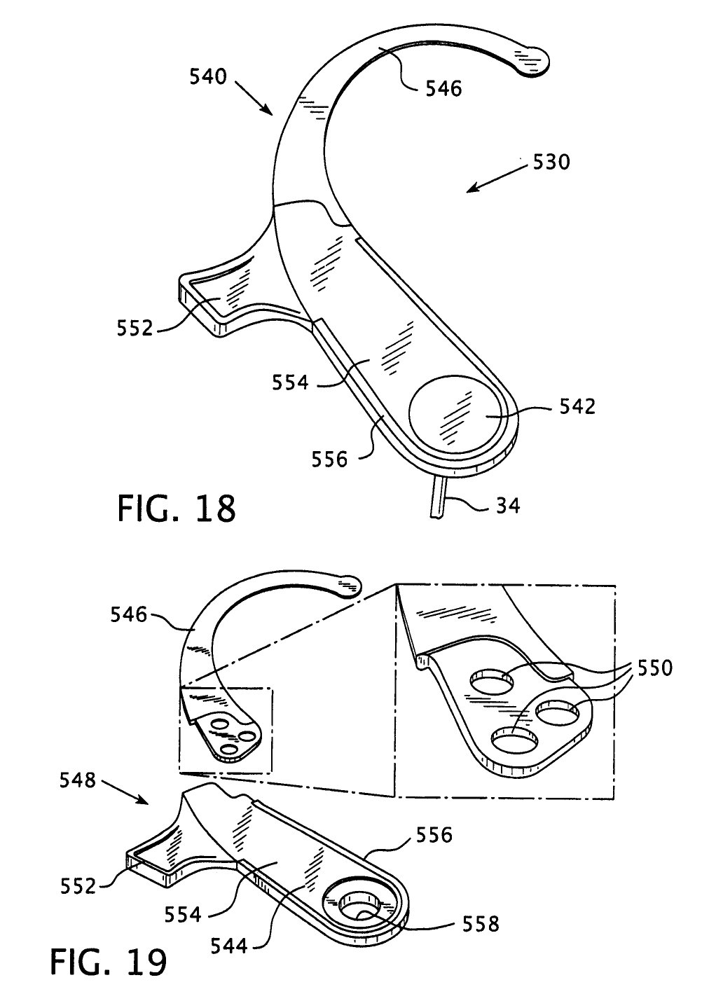

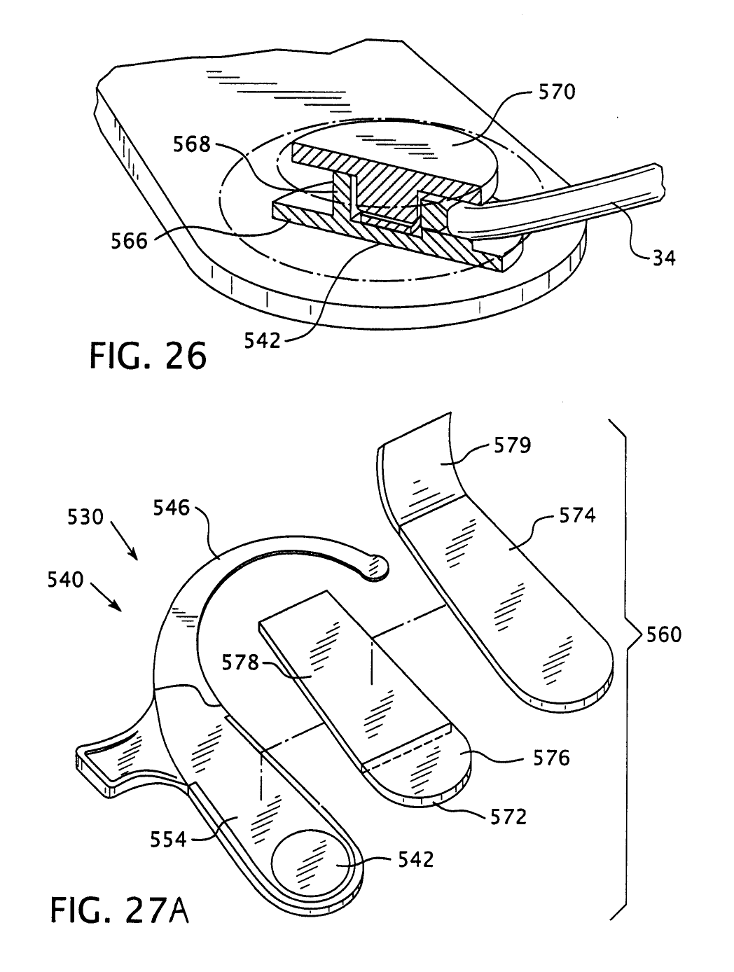

FIGS. 18-28 illustrate a sixth embodiment for electrode assembly 530 according to the principles of the present invention. Electrode assembly 530 includes a body member 540 and an adhesive assembly 560 that selectively attaches to the body member. Body member 540 includes an electrode support portion 544 and a curved portion 546. As in the previous embodiment, the present invention contemplates forming curved portion formed from a hard, molded plastic, such as the Polycarbonate GE Lexan HP 1. In this exemplary embodiment, electrode support portion 544 and curved portion 546 are formed from different components that are coupled together by an attachment assembly 548, which is best shown in FIG. 19.

Attachment assembly 548 includes a mechanism for coupling electrode support portion 544 to curved portion 546 in either a fixed (permanent) connection or a separable connection. In the illustrated embodiment, this mechanism in attachment assembly 548 includes a plurality of female opening 550 provided in curved portion 546 and matching male protrusions (not shown) provided in electrode support portion 544. The male protrusion engage the female opening to secure the curved portion of the body member to the electrode support portion. It is to be understood that other techniques for securing the curved portion of the electrode support portion are contemplated by the present invention, including adhesive boding, sonic welding, or a snap-fit assembly.

Electrode support portion 544 includes an optional extension 552 that extends from a side of the electrode assembly. This extension allows the user to grip the electrode assembly more easily as he or she inserts it against the back of the ear or removes it after use. Electrode support portion 544 also includes a first area 554 in which adhesive assembly 560 is disposed. A lip 556 is provided around at least a portion of first area. Lip 556 helps the user properly position the adhesive assembly in the first area by providing a small recess or cavity in which the adhesive assembly is located.

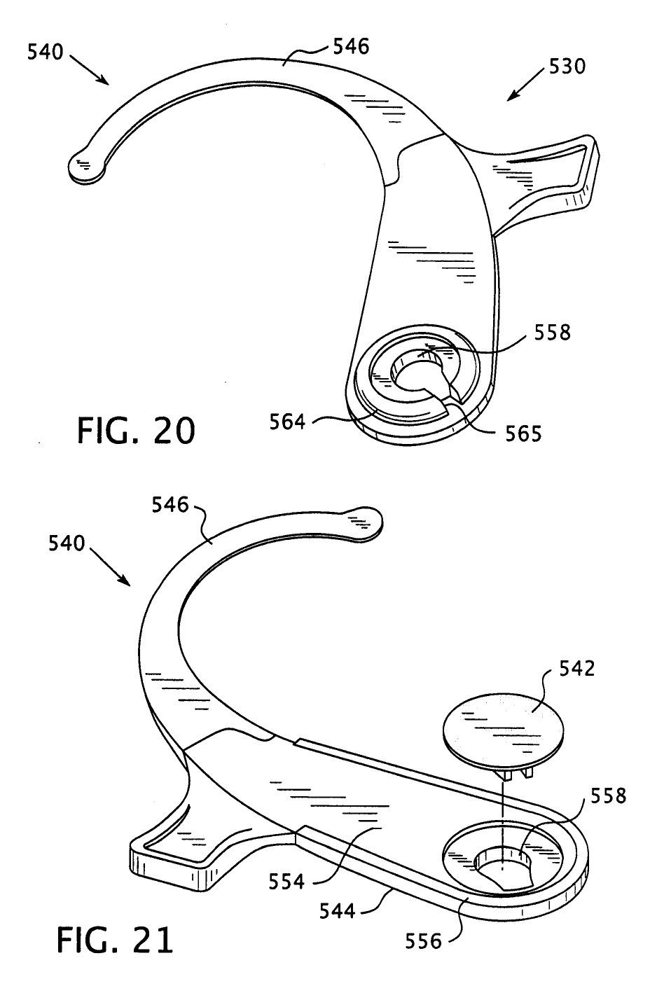

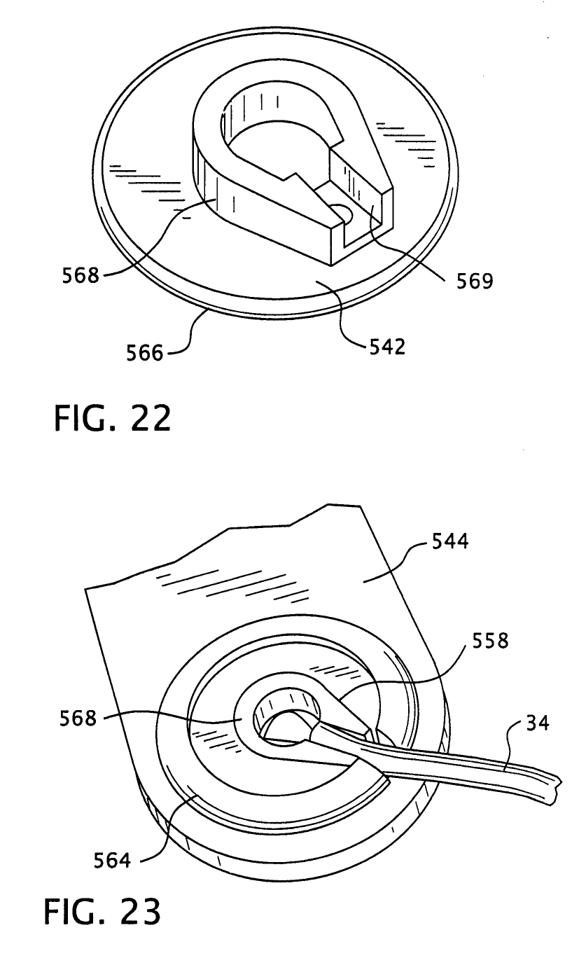

Finally, an opening 558 is provided in electrode support portion 544 to receive an electrode 542, which is illustrated in detail in FIG. 22. Opening 558 is sized, configured, and arranged to generally correspond to the size and shape of at least a portion the electrode and serves to define the position of the electrode on electrode assembly 530 relative to the other components of the electrode assembly. FIG. 20 shows opening 558 and a small hump or protrusion 564 provided on the exposed or non-user contacting surface of electrode support portion 544. A slot 565 is provided through a wall of protrusion.

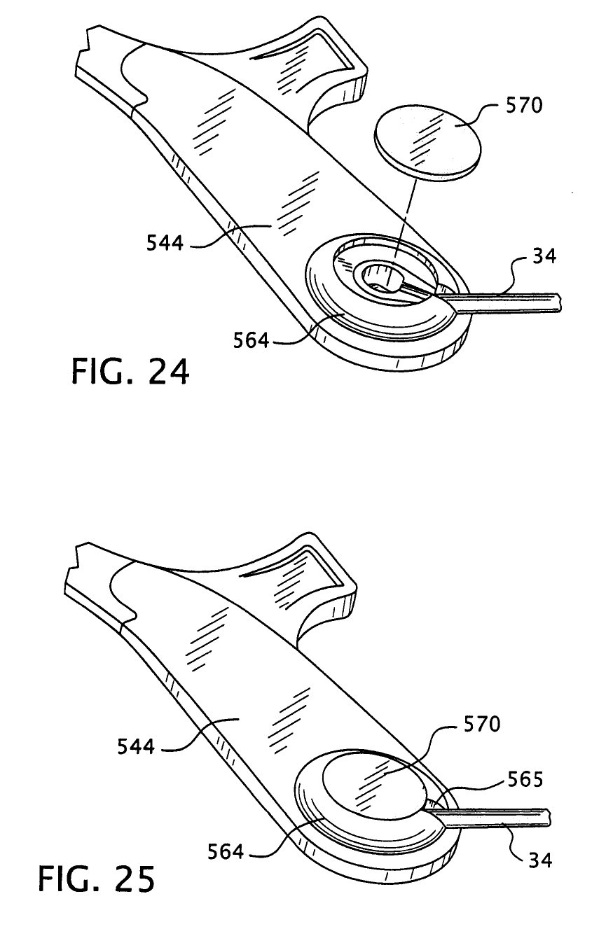

FIG. 21 illustrates how electrode 542 is inserted into opening 558, and FIGS. 21-23 shows details of the electrode and the attachment of the electrode to electrode support portion 544. Electrode 542 includes a generally flat or planer base 566 and a neck portion 568 that is in the form of a key-hole shape. Opening 558 generally matches the key-hole shape of neck 568 so that when the electrode is inserted into the opening the neck lies within key-hope shaped opening 558. Slot 565 provides access for electrical lead 34 to make electrical contact with the neck portion of electrode 542. A slot 569 is provided in neck 568 to receive electrical lead 34. As shown in FIGS. 24-26 a cover 570 is provided over neck 542 to crimp the electrical lead onto electrode 542. When in place, cover 570 is flush with the top of protrusion 564 and electrical lead 34 is securely fastened to the electrode lying under the cover.

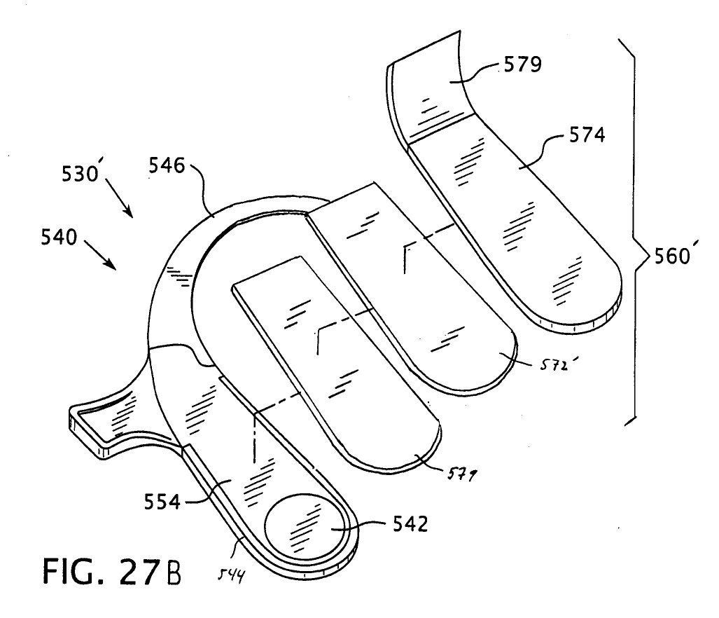

FIG. 27A illustrates an exemplary embodiment of adhesive assembly 560 provided over first area 554 of electrode support portion 544. In this embodiment, adhesive assembly 560 includes a hydrogel element 572 and a backing 574. Hydrogel element 572 includes a hydrogel layer 576 and a conductive portion 578. Conductive portion 578 is an electrically conductive element or material disposed in or on the hydrogel layer. An example of a conductive portion is a conductive backing provided on a portion of the hydrogel layer so at to abut electrode 542 when the adhesive assembly is attached to the electrode support portion. During use, adhesive assembly 560 is situated on electrode support portion 544 such that at least a portion of conductive portion 578 is in electrical contact with electrode 542. Backing 574 includes a tab 579 that extends from hydrogel element 572 so that the backing can be easily gripped and peeled off of the hydrogel element.

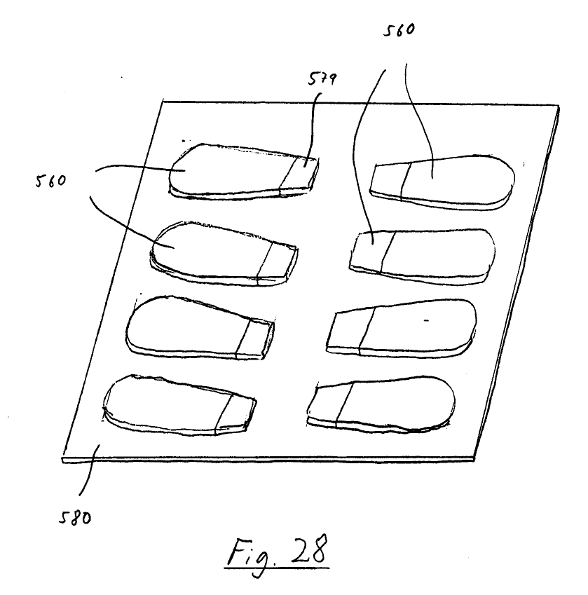

This embodiment is advantageous in that most of the electrode assembly can be reused. The only portion that needs to be replaced periodically is adhesive assembly 560. This is made simple by providing a plurality of adhesive assemblies 560 attached to a backing sheet 580, as shown in FIG. 28. The user removes one of the adhesive assemblies from the backing sheet by gently pulling on backing 574 so that both the hydrogel element 572 and the backing, i.e., the entire adhesive assembly, is removed from backing sheet 580. The user then applies the adhesive assembly to first area 554 of electrode support portion 544 by placing the surface of also hydrogel element 572 that was adjacent the backing sheet in contact with the surface of first area 554. Conductive portion 578 must make electrical contact with the electrode. The other surface of hydrogel element 572 remains covered by backing 574 until the user is ready to use the electrode assembly. When desired, the user removes backing 574 from hydrogel element 572 and applies the electrode assembly behind the ear so that the electrode is proximate to the mastoid, i.e., the vestibular system.

After use, the user removes adhesive assembly 560 from first area 554 of electrode support portion 544, for example by peeling the hydrogel element 572 off of the body member. The electrode support portion 544 and/or the entire body member can be cleaned and reused with a new adhesive assembly 560.

FIG. 27B illustrates a slight variation for an electrode assembly 530′ and an adhesive assembly 560′ suitable for use in the electrode assembly. This embodiment is similar to that of FIG. 27A, except that an electrode layer or film 579 is provided over first area 554 of electrode support portion 544. In an exemplary embodiment, electrode layer 579 is a conductive strip or tape that is adhered to electrode support portion 544. In a further exemplary embodiment, electrode layer 579 is not readily removable, i.e., is permanently coupled to electrode support portion 544. Adhesive assembly 560′, which is removably disposed over at least a portion of electrode layer 579, includes a hydrogel element 572′ and a backing 574.

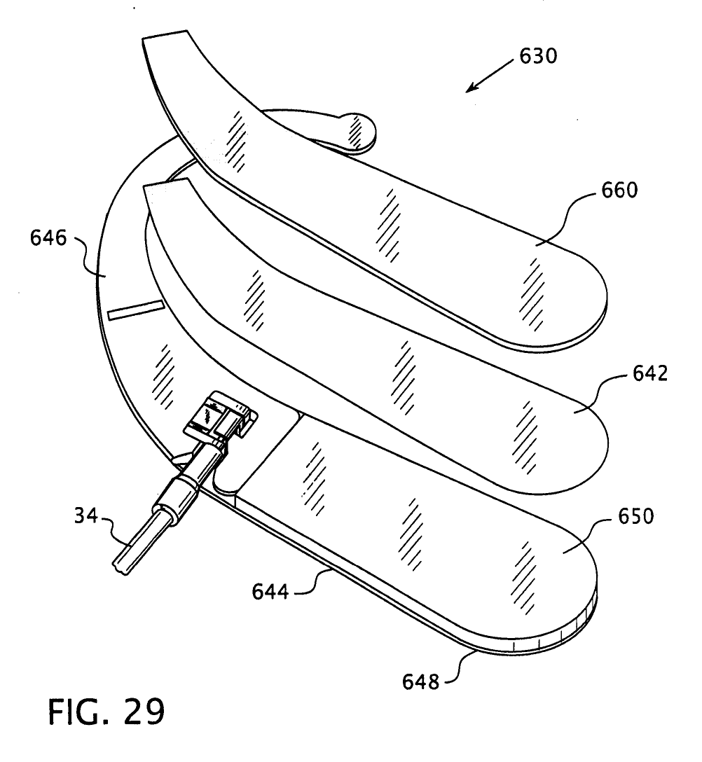

FIGS. 29-32 illustrate a seventh embodiment of an electrode assembly 630 according to the principles of the present invention. Electrode assembly 630 includes a body member 640 and an adhesive assembly 660 that selectively attaches to the body member. Body member 640 includes an electrode support portion 644 and a curved portion 646. Curved portion 646 is generally similar to curved portion 246 of the body member in the electrode assembly of FIGS. 8-14. An electrical lead 34 couples to curved portion 646 via a connection terminal provided in a slot provided in the curved portion. This connection is similar to that discussed above with respect to the electrode assembly of FIGS. 8-14. Thus the details for attaching the electrical lead to the body member are omitted for the sake of brevity.

Electrode support portion 644 includes a support member or layer 648 and a padding member 650. Support member 648 is coupled to curved portion 646 using any convention technique, such as an adhesive. Padding 650 is also coupled to support member 648 using any conventional technique, such as an adhesive. Support member 648 is made from a semi-rigid material or combination of materials. Padding 650 is any flexible, soft material, such as silicone or foam.

An electrode 642 is provided over electrode support portion 644. In this illustrated exemplary embodiment, the electrode also extends over a portion of curved portion 646. Electrode 642 is a flexible conductive layer or strip that is coupled to these portions of the electrode assembly, either permanently or in such as manner so that it can be removed from the other portions of the body member.

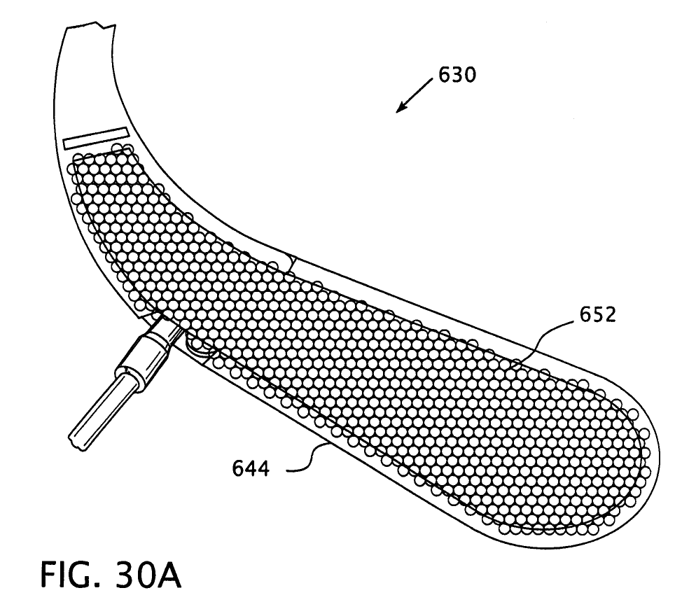

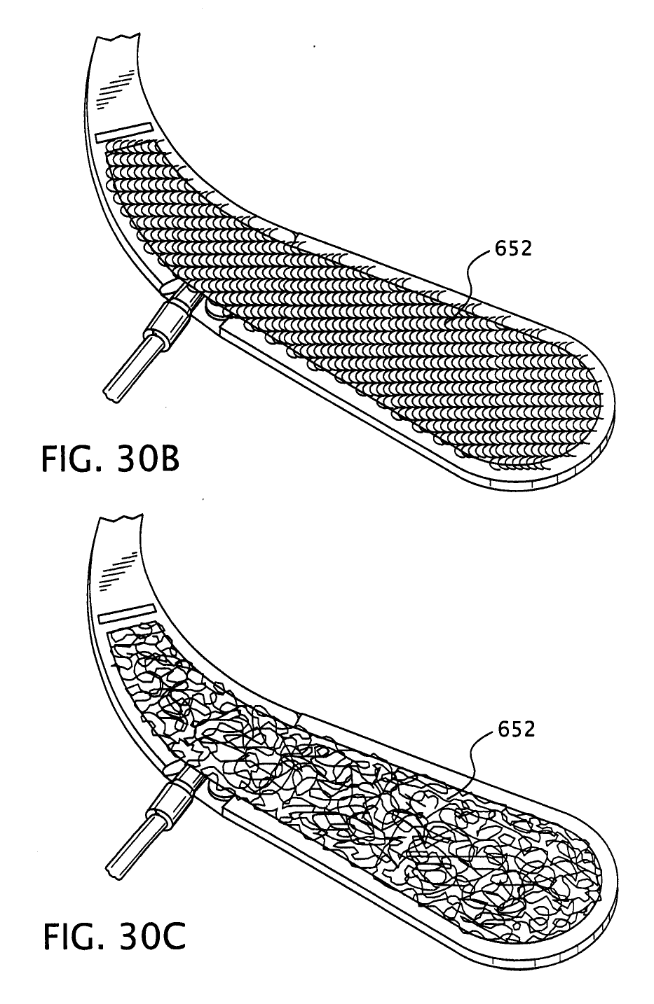

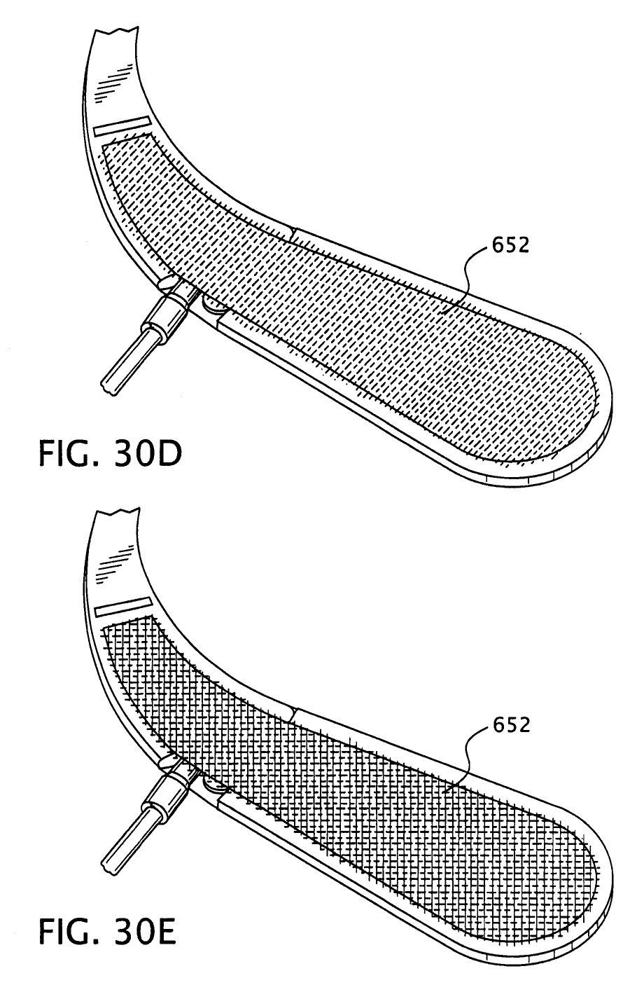

As shown in FIGS. 30A-30E, an insulating layer 652 is provided over all or a portion of electrode 642. In one embodiment, insulating layer 652 is a pattern of material that is printed or coated over the conductive layer. Insulating layer 652 can also be disposed on either surface of adhesive assembly 660, in the adhesive assembly, or any combination thereof. Insulating layer 652 serves to diffuse or disperse the electrical field so that the application of a stimulating current to the surface of the user is not concentrated at a location. When the current is concentrated at a location it tends to cause an undesirable or even possibly painful sensation in the surface of the user. The insulating layer prevents the current from being concentrated too much at one location, thereby diffusing the electrical energy delivered to the user, maximizing the comfort of the stimulation therapy provided to the user.

Insulating layer 652 can have a variety of different patterns so long as the goal of diffusing the electrical energy delivered to the user is accomplished. In the embodiment shown in FIG. 30A, the insulating layer is configured as a multitude of rings or circles connected edge-to-edge. FIGS. 30B-30E illustrate different embodiments, i.e., configurations, for the insulating layer. In these illustrated exemplary embodiments, the insulating layer is a series of waves (FIGS. 30B, 30D), a linked strands, (FIG. 30E) or a random dispersion of the insulating fibers (FIG. 30C).

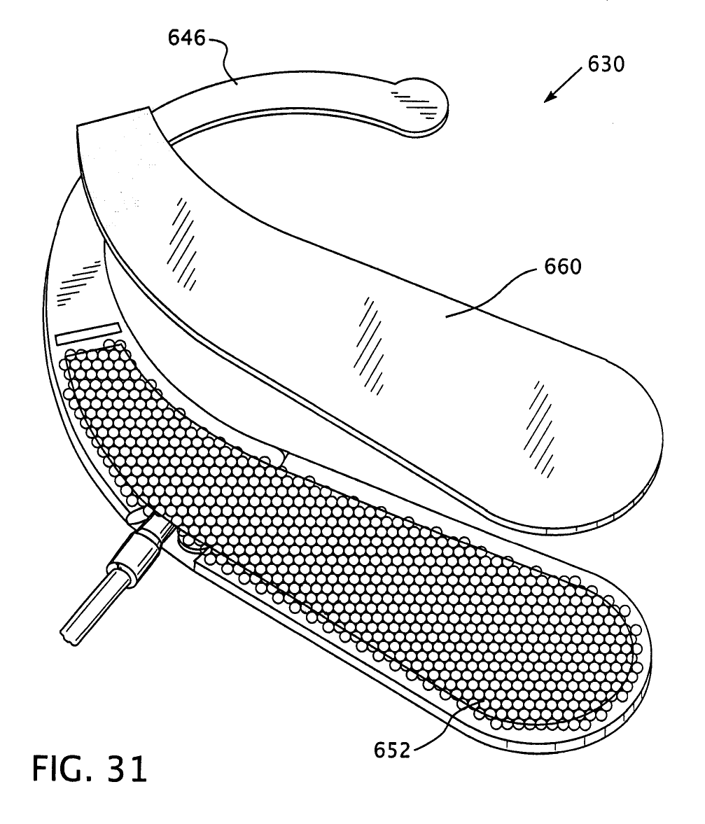

As shown in FIG. 31, adhesive assembly 660 is provided over insulating layer 652 to attach the electrode assembly to the user. In this embodiment, adhesive assembly 660 is a hydrogel layer or element. As in the previous embodiments, the adhesive assembly can include a backing (not shown) that is peeled away from the hydrogel to expose the hydrogel for use.

In the embodiments of FIGS. 30A-31, the insulating later is formed on or over the electrode. However, the present invention also contemplates providing the insulating layer as part of the adhesive assembly that is selectively attachable to the body member to form the complete electrode assembly. In an exemplary embodiment, the insulating layer is disposed on the hydrogel, for example, as a layer of insulation disposed over or under the hydrogel layer. The present invention also contemplates imbedding the insulating material in the hydrogel material.

It should again be emphasized that the features of the various embodiments of the present invention can be combined. For example, a strip electrode, such electrodes 242 or 642, and be provided over electrode 542 in the embodiment of FIGS. 18-27. An adhesive assembly that includes a hydrogel and an insulting layer 652 can be provided over this strip electrode to disperse the electrical energy delivered to the user. The resulting electrode has a body member 540, an electrode 242 or 642, and an adhesive assembly 660 (with an insulating layer). In addition, the present invention completed that the electrode assembly can be used to mount more than on electrode on the user by providing multiple electrodes on the electrode assembly.

The present invention further contemplates providing one or more sensors on the electrode assembly. For example, a temperature sensors, motion sensors, microphone, oximetry sensor, or any combination thereof can be provided for detecting various physiologic parameters of the user. The output of these sensors can be used to control the stimulation therapy provided by the user, stored for monitoring purposes, transmitted to a remote location, or any combination thereof.

The electrode assembly as stimulation system of the present invention can be used to stimulate other portions of the patient. As a simulation apparatus, the present invention could be used to stimulate the facial nerves, skin nerves, carotid sinus area verves (e.g. Herring Nerve), or any other nerve on the head or neck that can use the ear spiral anchor point as a point of reference. Stimulation energy can also be applied in a general manner, such as to awake or arouse the user from sleep or drowsiness, or as a tactile feedback function, for example, during an interactive game or simulation.

The present invention further contemplates that the electrode assemblies of the present invention need not be used to deliver energy, but can be used to monitor one or more of a variety of physiological parameters of the patient, such as electro-physiological impedance signals, physiological resistance, and the like. This can be done alone, i.e., without also delivering energy to the user, or in combination with an energy delivery function. Other parameters that can be monitored by the electrode assembly of the present invention include galvanic skin response and ear-to-ear impedance changes. The electrode assembly can also provide a reference platform for monitoring electro-physiological signals near the ear (even temple or forehead).

In the illustrated embodiment, an electrical lead is used to deliver energy to the electrode assembly. The present invention also contemplates eliminating the electrode making the electrode assembly wireless. In which case, a power source will be needed to provide energy to the electrode. Also a transceiver may be needed to control the deliver of the stimulating energy.

It can be appreciated from the above description of the present invention that this invention enables the adhesive assembly, i.e., the hydrogel, to be quickly and easily removed by from the electrode assembly so that it can be replaced by fresh hydrogel when needed. Most of the body member and the electrical lead of the electrode assembly is reused over a long period of time, while the removable piece, i.e., the adhesive member, is either a use-once throw-away, or is a convenient reusable storage cartridge which the user prepares well in advance of need.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. An electrode assembly for delivering electrical stimulation to a user, monitoring a physiological parameter or such as user, or both, the electrode assembly comprising:
(a) a body member comprising an electrode support portion and a curved portion extending from the electrode support portion; and
(b) an electrode coupled to the electrode support portion.

2. The apparatus of claim 1, further comprising an adhesive member coupled to or disposed over the body member, the electrode, or both.

3. The electrode assembly of claim 2, further comprising a removable backing disposed over at least a portion of the adhesive member.

4. The electrode assembly of claim 2, wherein the adhesive member is defined at least in part by a hydrogel, a hydrogel element, a foam, a tape, or any combination thereof.

5. The electrode assembly of claim 2, wherein the adhesive member is disposed on the body member over a first area, and wherein the body member further comprises a lip surrounding at least a portion of the first area.

6. The electrode assembly of claim 2, wherein at least a portion of the adhesive member is disposed over the electrode, and wherein at least a portion of the portion of the adhesive member disposed over the electrode is electrically conductive.

7. The electrode assembly of claim 1, wherein the curved portion has a generally spiral configuration or a shape that generally corresponds to a perimeter of a back of an external portion of a human ear.

8. The electrode assembly of claim 1, wherein at least a portion of the curved portion is plastic, paper, rubber, silicon, or any combination thereof.

9. The electrode assembly of claim 1, further comprising an insulating layer adapted to be coupled to the electrode support portion of the body member, and wherein the electrode comprises a conductive material or layer disposed on or in the insulating layer.

10. The electrode assembly of claim 1, wherein the electrode is adapted to be selectively coupled to the electrode support portion of the body member.

11. The electrode assembly of claim 10, further comprising a support member coupled to the electrode, wherein the electrode support portion of the body member includes an electrode support coupler, and wherein the support member includes an engagement portion adapted to be coupled to the electrode support coupler to attach the electrode to the body member.

12. The electrode assembly of claim 11, further comprising a hydrogel or a hyrogel element disposed on at least a portion of the electrode, the support member, or both.

13. The electrode assembly of claim 11, wherein the electrode is a substantially flat strip disposed in a surface of the support member.

14. The electrode assembly of claim 11, further comprising an insulating layer adapted to be coupled to the support member, and wherein the electrode is disposed on or in the insulating layer.

15. The electrode assembly of claim 1, further comprising a hydrogel element adapted to be disposed over at least a portion of the electrode.

16. The electrode assembly of claim 15, wherein the hydrogel element comprises:
a hydrogel material; and
a insulating material disposed on or in the hydrogel material.

17. The electrode assembly of claim 16, further comprising a backing material disposed over at least a portion of the hydrogel element.

18. The electrode assembly of claim 1, further comprising
(c) a source of stimulating energy; and
(d) a controller adapted to control delivery of the stimulating energy to the electrode.

19. The electrode assembly of claim 18, wherein the source of stimulating energy is (1) disposed on by the body member or (2) remote from the electrode.

20. An electrode assembly for delivering electrical stimulation to a user, monitoring a physiological parameter or such as user, or both, the electrode assembly comprising:
stimulating means for providing electrical stimulation to a user;
supporting means for securing the stimulating means to the user such that at least a portion of the supporting means wraps at least a portion of a perimeter at a back of the human ear.

21. The electrode assembly of claim 20, further comprising securing means for adhering the stimulating means, the supporting means, or both to a surface of a user.

22. The electrode assembly of claim 21, wherein the securing means comprises a replaceable element adapted to be selectively attached to the stimulating means, the supporting means, or both.

23. The electrode assembly of claim 20, wherein the stimulating means includes means for diffusing energy provided to such a user.

24. The electrode assembly of claim 20, further comprising attaching means for selectively attaching the stimulating means to the supporting means.

25. The electrode assembly of claim 20, further comprising:
energy supplying means for providing energy to the stimulating means; and
controlling means for controlling delivery of the electrical stimulation by controlling the energy supplying means.

26. A method of delivering electrical stimulation to a vestibular system of a user, comprising:
(a) securing an electrode to a surface of a user by wrapping at least a portion of an electrode support portion of a body member around at least a portion of a perimeter at a back of the human ear such that an electrode is disposed proximate to a mastoid; and
(b) providing energy to the electrode.

27. The method of claim 26, further comprising adhering the electrode to a surface of a user.

28. The method of claim 26, further comprising diffusing energy provided to such a user by the electrode.

29. A method of delivering electrical stimulation to a user, comprising:
providing an electrode assembly;
applying a hydrogel element to a portion of the electrode assembly;
affixing the electrode assembly to a user;
using the electrode assembly to deliver energy to such a user;
removing the hydrogel element from the electrode assembly;
applying a subsequent hydrogel element to the portion of the electrode assembly; and
reusing the electrode assembly to deliver energy to such a user.

30. The method of claim 29, wherein using the electrode assembly to deliver energy to a user includes affixing the electrode assembly to a surface of a user by contacting the hydrogel with the surface of the user.

31. The method of claim 29, wherein applying the hydrogel element to the portion of the electrode assembly includes:
selecting a hydrogel assembly that includes the hydrogel element and a backing disposed on at least a portion of the hydrogel element;
applying the hydrogel assembly to the portion of the electrode assembly; and
removing the backing before affixing the electrode assembly to a user.

32. An electrode assembly, comprising:
(a) a body member;
(b) an electrode coupled to the body member; and
(c) a hydrogel element disposed on at least a portion of the electrode, wherein the hydrogel element comprises:
(1) a hydrogel material; and
(2) a insulating material disposed on or in the hydrogel material such that the insulating layer disperses energy transmitted by the electrode.

33. The electrode assembly of claim 32, further comprising a removable backing disposed over at least a portion of the hydrogel element.

34. The electrode assembly of claim 32, wherein the insulating material is disposed in or on the hydrogel material.

35. The electrode assembly of claim 32, wherein the hydrogel element is disposed on the body member over a first area, and wherein the body member further comprises a lip surrounding at least a portion of the first area.
